(19) United States
(12) Patent Application Publication
(10) Pub. No.: US 2022/0091568 A1
(43) Pub. Date: Mar. 24, 2022

(54) METHODS AND DEVICES FOR PREDICTING PHYSICAL PARAMETER BASED ON INPUT PHYSICAL INFORMATION

(71) Applicant: SHENZHEN KEYA MEDICAL TECHNOLOGY CORPORATION, Shenzhen (CN)

(72) Inventors: Bin Kong, Charlotte, NC (US); Youbing Yin, Kenmore, WA (US); Xin Wang, Seattle, WA (US); Yi Lu, Seattle, WA (US); Qi Song, Seattle, WA (US)

(73) Assignee: SHENZHEN KEYA MEDICAL TECHNOLOGY CORPORATION, Shenzhen (CN)

(21) Appl. No.: 17/468,040

(22) Filed: Sep. 7, 2021

Related U.S. Application Data
(60) Provisional application No. 63/081,279, filed on Sep. 21, 2020.

(51) Int. Cl. G05B 13/04; G05B 13/02

(52) U.S. Cl. CPC G05B 13/042; G05B 13/0265; G05B 13/048

(57) ABSTRACT

The disclosure relates to a method and device for predicting a physical parameter based on input physical information, and medium. The method may include predicting, by a processor, an intermediate variable based on the input physical information with an intermediate sub-model, which incorporates a constraint on the intermediate variable according to prior information of the physical parameter. The method may also include transforming, by the processor, the intermediate variable predicted by the intermediate sub-model to the physical parameter with a transformation sub-model.

### CROSS REFERENCE TO RELATED APPLICATION

This application is based on and claims the benefit of priority of U.S. Provisional Application No. 63/081,279, filed on Sep. 21, 2020, which is incorporated herein by reference in its entirety.

### TECHNICAL FIELD

The present disclosure relates to physical parameter prediction using machine learning, and more specifically, to methods and devices for predicting physical parameter using prior information of physical information as a constraint, such as predicting a fractional flow reserve (FFR) value of a blood vessel.

### BACKGROUND

Machine learning has been used as an essential tool to model complex functions across many domains, such as insurance (insurance premium prediction), healthcare (medical diagnosis, development, and growth), agriculture (plant growth), etc. With the increased complexity of the learning model, it is able to improve the prediction ability for various complex problems in real practice. However, since the learning model is mainly configured to deduce a mapping function (as a black box) from the input physical information to the output physical parameter based on training data, the predicted results may not obey the fundamental rules that govern the physical parameters. As an example, the insurance premium predicted by the learning model may decrease with the age (which contradicts with the fundamental rule that the insurance premium will increase with the age). As another example, the height of a child predicted by the learning model may decrease as the child grows up (which contradicts with the fundamental rule that the height of a child should be growing). As another example, the pressure of blood flow predicted by the learning model may be increasing from upstream to downstream in vessel trees (which contradicts with the fundamental rule that the pressure of blood flow is decreasing from upstream to downstream in vessel trees).

To compensate for the fact that the fundamental rule governing the physical parameter to be predicted is usually ignored by the learning models, some conventional methods consider the fundamental rule related information through post-processing steps. However, these methods require additional steps and these steps decrease the performance of the learning model. Some other methods may use additional loss term(s) in the loss function designed to penalize predications during the training stage which contradict with the fundamental rule. Taking the monotonic profile of the physical parameters in sequence as an example, an addition loss term designed to penalize the non-monotonic predictions is adopted in the loss function during the training stage. However, a low non-monotonic loss in the training data does not necessarily mean a low non-monotonic loss for all testing data, especially when the model is overfitting the training data. More importantly, it does not guarantee that the predictions are strictly monotonic.

There is still room to improve the learning model, especially for those intend to model complex functions with prior information.

### SUMMARY

The present disclosure is provided to solve the above-mentioned problems existing in the prior art. There is a need for methods and devices for predicting physical parameter based on the input physical information by means of a learning model, and computer-readable media, which may enforce the prior information of the physical parameter as a constraint function into the architecture of the learning model, without requiring additional loss terms or post-processing steps. Accordingly, the prediction result could be forced substantially comply with the fundamental rule, thus the model performance could be improved.

According to a first aspect of the present disclosure, a method for predicting a physical parameter based on input physical information is provided. The method may include predicting, by a processor, an intermediate variable based on the input physical information with an intermediate sub-model, which incorporates a constraint on the intermediate variable according to prior information of the physical parameter. The method may also include transforming, by the processor, the intermediate variable predicted by the intermediate sub-model to the physical parameter with a transformation sub-model.

According to a second aspect of the present disclosure, a device for predicting a physical parameter based on input physical information is provided. The device may include a storage and a processor. The storage may be configured to load or store an intermediate sub-model and a transformation sub-model. The processor may be configured to predict an intermediate variable based on the input physical information with the intermediate sub-model, which incorporates a constraint on the intermediate variable according to prior information of the physical parameter, and transform the intermediate variable predicted by the intermediate sub-model to the physical parameter with the transformation sub-model.

According to a third aspect of the present disclosure, a non-transitory computer-readable medium is provided with computer-executable instructions stored thereon. The computer-executable instructions, when executed by a processor, may perform a method for predicting a physical parameter based on input physical information. The method may comprise predicting an intermediate variable based on the input physical information with an intermediate sub-model, which incorporates a constraint on the intermediate variable according to prior information of the physical parameter. The method may further comprise transforming the intermediate variable predicted by the intermediate sub-model to the physical parameter with a transformation sub-model.

The above method and device, as well as the medium, may enforce the prior information of the physical parameter as a constraint function into the architecture of the learning model, without requiring additional loss terms or post-processing steps, to guarantee that the prediction result substantially comply with the fundamental rule and improve the model performance.

The foregoing general description and the following detailed description are only exemplary and illustrative, and do not intend to limit the claimed invention.

### BRIEF DESCRIPTION OF THE DRAWINGS

In the drawings that are not necessarily drawn to scale, similar part numbers may describe similar components in different views. Similar part numbers with letter suffixes or different letter suffixes may indicate different examples of similar components. The drawings generally show various embodiments by way of example and not limitation, and together with the description and claims, are used to explain the disclosed embodiments. Such embodiments are illustrative and exemplary, which are not intended to be exhaustive or exclusive embodiments of the method, system, or non-transitory computer-readable medium having instructions for implementing the method thereon.

FIG. 1 illustrates a schematic diagram of an exemplary framework of a physical parameter prediction model, according to an embodiment of the present disclosure.

FIG. 2 shows a flowchart of an exemplary method for predicting a physical parameter based on input physical information, according to an embodiment of the present disclosure.

FIG. 3 illustrates a flowchart of an exemplary method of training the physical parameter prediction model, according to an embodiment of the present disclosure.

FIG. 4 illustrates a schematic diagram of an exemplary physical parameter prediction model, according to an embodiment of the present disclosure.

FIG. 5 illustrates a schematic diagram of another exemplary physical parameter prediction model, according to an embodiment of the present disclosure.

FIG. 6 illustrates a schematic block diagram of an exemplary device for predicting a physical parameter based on input physical information, according to an embodiment of the present disclosure.

FIG. 7 illustrates a schematic block diagram of an exemplary system for predicting a physical parameter based on input physical information, according to an embodiment of the present disclosure.

### DETAILED DESCRIPTION

Reference will now be made in detail to exemplary embodiments, examples of which are illustrated in the drawings.

In this disclosure, “physical information” may be any information which may be collected or acquired in various technical domains that is governed by certain physical rules. The physical information may be acquired in various formats, such as but not limited to a sequence of data, vectors, image patches, list, etc. Correspondingly, “physical parameter” to be predicted may be a physical parameter related to the physical information in the corresponding technical domain. For example, in the technical domain of insurance, the age and healthcare information of the insured object may be adopted as the physical information, and the insurance premium of the insured object may be set as the physical parameter to be predicted. As another example, in the technical domain of healthcare, such as coronary artery stenosis diagnosis, the sequence of image patches in a coronary artery tree may be adopted as the physical information, and the sequence of fractional flow reserve (FFR) or instantaneous wave-free ratio (iFR) in the coronary artery tree may be set as the physical parameter to be predicted. In this disclosure, “prior information of the physical parameter” may comprise known or confirmed knowledge related to the predicted physical parameters, such as the fundamental rule(s) that govern the physical parameter or its transformed parameter according to a physical principle or theory. In the exemplary technical domain of insurance, an example of the prior information may be that the insurance premium has to increase with the insurer's age increasing and the healthcare condition getting worse. In the exemplary technical domain of coronary artery stenosis diagnosis, an example of the prior information may be that FFR values from downstream should not be higher than the ones from upstream of the coronary artery trees.

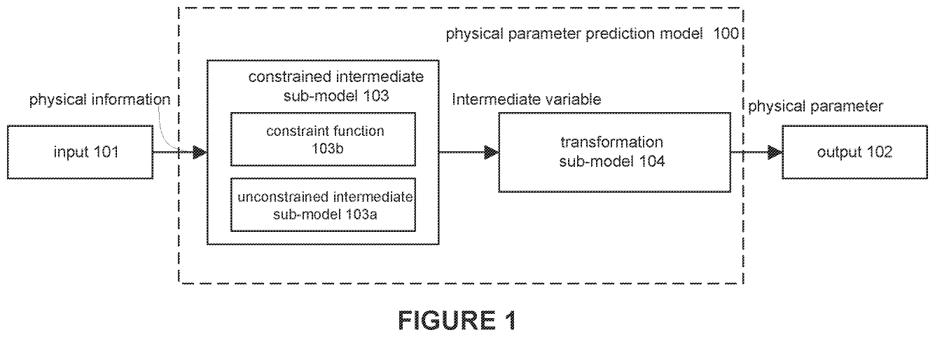

FIG. 1 illustrates a schematic diagram of an exemplary framework of a physical parameter prediction model 100, according to an embodiment of the present disclosure. The physical parameter prediction model 100 may model a predetermined relationship between a physical parameter and input physical information, e.g., the physical parameter being a target function of the physical information. Instead of directly modeling the target function, as shown in FIG. 1, the physical parameter prediction model 100 may be divided generally into two sub-models: one is a constrained intermediate sub-model 103 and the other is a transformation sub-model 104. The constrained intermediate sub-model 103 may be configured to receive physical information as input 101, where the physical information may be acquired from a particular technical domain. The constrained intermediate sub-model 103, when applied by a processor, may be configured to predict an intermediate variable based on the received physical information, and the prediction can be regulated by a constraint complying with prior information governing the technical domain in which the physical information is acquired. The transformation sub-model 104 then maps the intermediate variable to the physical parameters. As a result, the physical parameter prediction model 100 can be applied to predict physical parameters from input physical information, with the prior information taken into consideration.

As shown in FIG. 1, the constrained intermediate sub-model 103 may comprise an unconstrained intermediate sub-model 103a and a constrain function 103b. The constrained intermediate sub-model 103 may incorporate a constraint, e.g., constraint function 103b, on the intermediate variable according to prior information of the physical parameter.

In some embodiments, the prior information of the physical parameter(s) may include a profile tendency (especially for the physical parameters as a sequence) and/or bound range (e.g., of the magnitude) (e.g., positive, negative, or within a range defined by a lower limit and/or an upper limit, etc.,) in temporal domain and/or spatial domain. In some embodiments, the profile tendency may include any one of monotonicity (e.g., increasing, decreasing, non-increasing, or non-decreasing) of profile change, periodicity of profile change, convex shape of the profile, and concave shape of profile for the sequence of physical parameters.

In some embodiments, the intermediate variable may be determined based on the prior information of the physical parameter(s) so that the prior information may be mathematically expressed by the intermediate variable as the constrain function 103b. Based on the prior information of the physical parameter(s), the intermediate variable may be pre-defined to model an intermediate function of the input physical information and the transformation sub-model 104 may be a function constructed according to the intermediate function and the target function, so that they collectively model the target function. As an example, when the prior information is an increasing monotonicity of the profile change of the sequence of physical parameters, the derivative of the physical parameter may be set as the intermediate variable, and a function mapping the derivatives of the physical parameter to positive values, such as but not limited to ReLU, may be adopted as the constraint function 103b as part of the intermediate sub-model 103. Accordingly, the transformation sub-model 104 may be set as an integral function (or based on the integral function).

In some embodiments of the present disclosure, for each prediction of the physical parameter(s), the intermediate variable(s) of the physical parameter(s) is first predicted without constrain conditions and then is treated directly by means of the constrain function 103b to satisfy the prior information. After that, inverse operation with respect to the operation for obtaining the intermediate variable(s) from the physical parameter(s) may be performed as the transformation sub-model 104 on the predicted intermediate variable. As a result, the prediction result of the physical parameter(s), e.g., the output 102, can be ensured to be consistent with the prior information. The resulted physical parameter predict model 100 may achieve an accurate prediction performance on the physical parameter(s) in an end-to-end manner (i.e., post-processing steps may not be needed), meanwhile efficiently suppressing unrealistic (contradicting with the prior information) data and preventing from overfitting the training data.

In some embodiments, for the sequence of physical parameters, the prior information may govern the whole sequence, partial segments, or sporadic locations/points in sequences, or samples in scalar prediction problems.

In some embodiments, the unconstrained intermediate sub-model 103a may be generated in various manners, including but not limited to, as a linear model, curve model (e.g., polynomial model), learning model (such as a machine learning model or a deep learning model), etc. In some embodiments, the unconstrained intermediate sub-model 103a may be configured as a learning model, such as a decision tree, support vector machine, Bayesian forecast model, CNN, or MLP, etc., to model hidden and complex mapping functions between the physical information (e.g., input 101) and the intermediate variable(s).

Generally, the present disclosure may relate to two phases: a prediction phase and a training phase. The training phase can be performed to train the physical parameter predict model 100 and the prediction phase can be performed to apply the trained physical parameter predict model 100 to make predictions of the physical parameter based on input physical information. Each of the prediction phase and the training phase can be performed online (e.g., in real time) or offline (e.g., in advance). In some embodiments, the training phase may be performed offline and the prediction phase may be performed online.

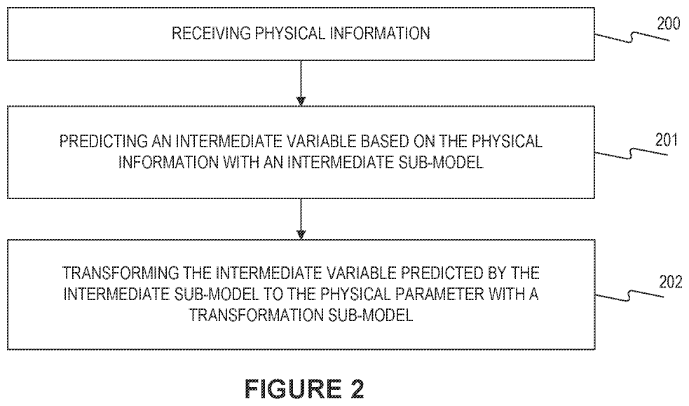

FIG. 2 illustrates a flowchart of an exemplary method for predicting physical parameter(s) based on input physical information, according to an embodiment of the present disclosure.

As shown in FIG. 2, the method begins with a step 200: receiving physical information. The physical information may be acquired in a specific technical domain. At step 201, the method may include predicting, by a processor, an intermediate variable based on the input physical information with an intermediate sub-model, which incorporates a constraint on the intermediate variable according to prior information of the physical parameter. At step 202, the method may further include transforming, by the processor, the intermediate variable predicted by the intermediate sub-model to the physical parameter with a transformation sub-model.

For example, the technical domain may be the medical field, and the physical information may be medical information, such as clinical information of the disease history, image(s) (or patches) and/or feature vector (either explicitly defined or hidden feature information) extracted therefrom. And the physical parameter(s) may be medical parameter(s) accordingly. For example, the medical parameter(s) may include a medical index, physiological status parameter, the diseased type, etc. The medical image(s) may be acquired via any image modality among the follows: functional MRI (e.g., fMRI, DCE-MRI and diffusion MRI), Cone Beam CT (CBCT), Spiral CT, Positron Emission Tomography (PET), Single-Photon Emission Computed Tomography (SPECT), X-ray, optical tomography, fluorescence imaging, ultrasound imaging, and radiotherapy portal imaging, etc., or the combination thereof.

The details of each of the intermediate sub-model, the constrain function, and the transformation sub-model have already been described with reference to FIG. 1, and thus are not repeated here.

In some embodiments, the constrained intermediate sub-model 103 may be a learning model (e.g., a machine learning model or a deep learning model), and the transformation sub-model 104 may be a preset function. In some embodiments, the constrained intermediate sub-model 103 and the transformation sub-model 104 may be collectively trained with training dataset of the physical information annotated with the physical parameter(s). In this manner, the lack of the ground truth labels of the intermediate variable(s) may be overcome, instead, the abundance of ground truth labels of the physical parameter(s) may be utilized to train the physical parameter predict model 100 as a whole. The training of the physical parameter predict model 100 effectively trains the constrained intermediate sub-model 103 as a learning model.

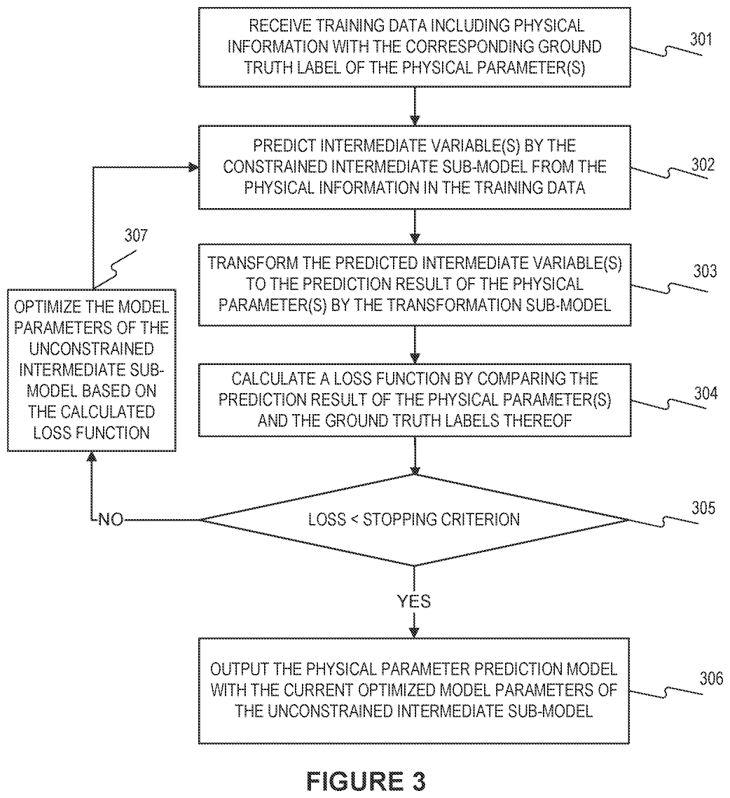

For the physical parameter prediction model with a predefined configuration, i.e., each of the intermediate variable(s), the transformation sub-model, and the constraint function are predefined, and the configuration of the unconstrained intermediate sub-model is predetermined (such as CNN), the training process may be performed as shown in FIG. 3.

The training process may begin with step 301, where training data including physical information and the corresponding ground truth labels of the physical parameter(s). The training data is input into the physical parameter prediction model (with predefined framework such as shown in FIG. 1). In some embodiments, the model parameters (such as weights) of the unconstrained intermediate sub-model within the physical parameter prediction model may be initialized. For example, the model parameters may be initialized as all 0s or 1s, or a set of values used in a previously trained intermediate sub-model (for the same technical domain or a different technical domain).

At step 302, from the physical information in the training data, intermediate variable(s) may be predicted by the constrained intermediate sub-model with the current model parameters. At step 303, the predicted intermediate variables are then transformed to the prediction result of the physical parameter(s) by the transformation sub-model. At step 304, the loss function may be calculated by comparing the prediction result of the physical parameter(s) and the ground truth labels thereof. At step 305, the calculated loss is compared to a stopping criterion, e.g., a nominal threshold value. If the calculated loss is below the stopping criterion (step 305: YES), the current model parameters are sufficiently optimized and no more iteration is necessary. Accordingly, the method proceeds to step 306, to output the physical parameter prediction model with the current model parameters of the unconstrained intermediate sub-model. Otherwise (step 305: NO), further optimization is needed. At step 307, the model parameters of the unconstrained intermediate sub-model may be optimized based on the calculated loss function. Then the method iterates steps 302-305 based on the updated unconstrained intermediate sub-model with the current model parameters, until the loss is less than the stopping criterion.

In some embodiments, the optimization process of the model parameters may be performed by various algorithms, such as but not limited to stochastic gradient descent method, Newton method, conjugate gradient method, Quasi-Newton Method, and Levenberg Marquardt algorithm, etc.

Since the prior information is enforced explicitly, through the constraint applied on the intermediate variable, the physical parameter prediction model does not require additional loss terms with respect to the prior information in the training process. Besides, the training process may guarantee that the prediction results comply with the prior information with workload comparable to the training process of other physical parameter prediction model that attempts to avoid overfitting efficiently without enforcing the prior information.

In some embodiments, the sequence of physical parameters may include vessel parameters at a sequence of positions in a vessel structure, such as a vessel tree or a vessel path.

Hereinafter, fraction flow reserve (FFR) is described as an example of the physical parameter(s). Two examples of prior information, i.e., monotonicity of profile change of a sequence of physical parameters and the bound range of a single physical parameter, are used to illustrate how to enforce explicitly various prior information into the physical parameter prediction model. However, these exemplary methods described for prediction of FFR may be applied or adapted to predict other medical or physiological parameters in the medical fields, or physical parameters in other technical fields. Besides, these methods may also be adapted to accommodate other types of prior information.

Fractional flow reserve (FFR) is considered to be a reliable index for the assessment of the cardiac ischemia and the learning models have been used to predict FFR values in the coronary artery tree. FFR is defined as a ratio between the pressure after a stenosis (or the pressure at any position within the vessel tree) and the pressure at the ostia point (the inlets of the coronary artery tree). Following the physics, in the sequence of FFR values within the coronary artery trees FFR values from downstream should not be higher than the one from upstream.

In some embodiments, instead of predicting FFR values directly, the methods and devices of present disclosure can be used to model the drop of FFR of the current point relative to the adjacent upstream point. The drop of FFR values may be defined as the derivative of the FFR along sequences. Based on the monotonicity of the profile change of sequence of FFR values along vessel structure, the intermediate variable may be defined based on derivative of the sequence of FFR values (such as derivative of the upstream FFR value with respect to its adjacent downstream FFR value), and correspondingly, the constraint function may be defined as mapping into non-negative range, the transformation sub-model may be defined based on an integral function to obtain the sequence of FFR values from the non-negative derivatives of the sequence of FFR values. Similarly, for other physical parameters with its prior information including the monotonicity of profile change of the sequence of physical parameters, the intermediate variable can be defined based on derivative of the sequence of physical parameters.

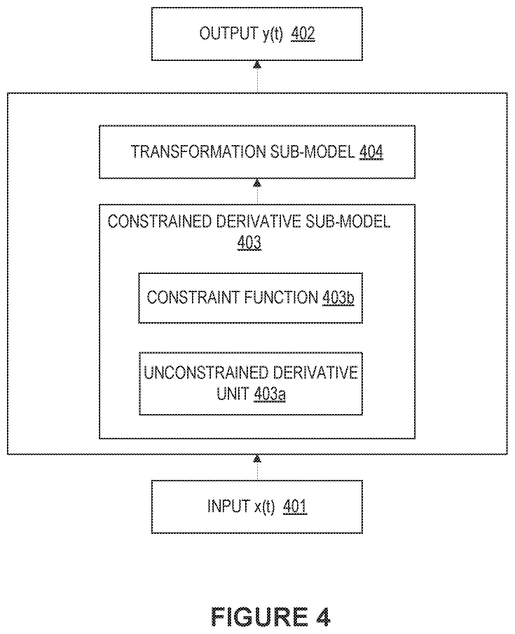

As shown in FIG. 4, the FFR prediction model may receive image patches or feature vectors along the coronary artery trees or paths as input 401 x(t). The FFR prediction model may include a constrained derivative sub-model 403 and a transformation sub-model 404.

The constrained derivative sub-model 403 aims to model the derivatives of the sequence of FFR values. Based on the predicted derivatives of the sequence of FFR values, the transformation sub-model 404 may map the constrained derivatives to the FFR values in the target domain.

As shown in FIG. 4, the constrained derivative sub-model 403 may include an unconstrained derivative unit 403a and a constraint function 403b, and may be based on a learning model (especially for the unconstrained derivative unit 403a). Particularly, the unconstrained derivative unit 403a may be constructed as a convolutional neural network (CNN), multi-layer perceptron (MLP), fully convolutional neural network (FCN), etc. The constraint function 403b may be implemented by an activation function at the end of the learning model for the unconstrained derivative unit 403a. In some embodiments, an activation function of ReLU may be adopted to force the drop of upstream FFR with respect to the downstream FFR to be non-negative, to incorporate the non-increasing FFR prior information into the FFR prediction model. It is contemplated that ReLU is only an example of the activation function, and other examples of activation functions, such as Sigmoid, etc., that can map the derivatives into a non-negative range, may also be adopted as appropriate.

The final predicted FFR values y(t) could be calculated from the output of the activation function, i.e., the non-negative derivatives of the sequence of FFR values, essentially the non-negative drop of sequence of FFR values along the vessel trees/paths, recursively using the transformation sub-model 404. Then the final predicted FFR values y(t) may be provided as output 402, as shown in FIG. 4.

As a result, it does not require additional loss terms to penalize the non-monotonic predictions as it can be enforced explicitly in the FFR prediction model.

In some embodiments, the FFR prediction model is designed to model a target function, i.e., the true underlying function F(x(t)). For example, the FFR prediction model can be expressed as a function ϕ(x(t)). ϕ(x(t)) is built to model the target function F(x(t)) with an intermediate function f(x(t)) (corresponding to the trained unconstrained derivative unit 403a). For example, the intermediate function f(x(t)) may be derivative functions of F(x(t)), wherein t denotes the position or index in sequences, the position may move toward the downstream as t increases. As an example, the intermediate function f(x(t)) may be defined as Formula 1 below:

$$f ( x ( t ) ) = \frac{\partial F ( x ( t ) )}{\partial t} , \qquad ( 1 )$$

or some other transform functions.

Based on the intermediate function f(x(t)), a function ϕ(x(t)) (corresponding to the trained FFR prediction model) may be built which tries to model and approximate the true underlying function F(x(t)).

As shown in FIG. 4, the input x(t) 401 may be fed firstly into the constrained derivative sub-model 403 Ø(.;θ), parameterized by θ. The constrained derivative sub-model 403 Ø(.;θ) may model the intermediate function f(x(t)), instead of the underlying function F(x(t)). Ø(x(t);θ) may be easily used to enforce the prior information, i.e., the constrained intermediate values predicted by Ø(.;θ) may be further fed into the transformation sub-model 404, yielding the final prediction result of FFRs y(t). Particularly, the input x(t) 401 may be firstly fed into the unconstrained derivative unit 403a, to predict the ‘raw’ (which does not undergo the verification of the prior information of non-decreasing monotonicity) FFR derivatives (of the upstream position to an adjacent downstream position) within the vessel tree. The ‘raw’ FFR derivatives as predicted are then fed into the constraint function 403b, e.g., an activation function of ReLU, which is connected at the end of the unconstrained derivative unit 403a. The constraint function 403b may map the ‘raw’ FFR derivatives to constrained (non-negative) FFR derivatives, to comply with the prior information of non-decreasing monotonicity from downstream to upstream. The non-negative FFR derivatives may be output by the constraint function 403b and fed into the transformation sub-model 404, to yield and output the final prediction result of FFRs y(t) 402, which are enforced to comply with the prior information of non-decreasing monotonicity of FFRs along the vessel tree from downstream to upstream, by the constraint function 403b in the constrained derivative sub-model 403.

The loss function L may be computed by comparing the yielded prediction result y(t) and the ground truth of the FFR. For a training set D, the parameter θ may be optimized by minimizing the loss function L. Methods such as stochastic gradient descent related methods may be used for optimization.

Without limiting the scope of disclosure, a type of the prior information of FFRs, i.e., non-decreasing monotonicity, may be used as an example throughout the descriptions. For example, the function ϕ(x(t)) may be a monotonic function by using derivative as the intermediate variable together with the non-negative constraint function 403b, which maps the input x(t) 401 to the output y(t) 402, such that y(t1)>y(t2) for any t2>t1. For different prediction problems, input x(t) may be an image or a feature vector. The constrained derivative sub-model 403 Ø(.;θ) may model the derivative function defined by Formula (1), instead of the underlying function F(x(t)). Ø(x(t)) may be easily constrained to be monotonic by enforcing the constrained derivative sub-model Ø(.;θ) to be non-negative (i.e., ensuring that the predicted FFR values are non-decreasing from downstream to upstream). In some embodiments, if the prior information requires non-increasing of the predicted values, the constrained derivative sub-model Ø(.;θ) may be enforced to be non-positive; if the prior information requires only increasing of the predicted values, the constrained derivative sub-model Ø(.;θ) may be enforced to be positive; if the prior information requires only decreasing of the predicted values, the constrained derivative sub-model Ø(.;θ) may be enforced to be negative. The so-predicted constrained derivatives may be fed into the transformation sub-model 404, yielding the final prediction result y(t), e.g., according to Formula (2) as follows:

y(t)=∫Ø(x(t);θ)dt (2)

If the prediction result y(t0) at a position t0 is given (either predefined or determined by a machine learning model), y(t0)=y0, the prediction result y(t) may be computed by the following Formula (3):

y(t)=y0+∫ₜ₀^{t}Ø(x(t);θ)dt (3)

Finally, a value of the loss function L may be computed by comparing the generated prediction result y(t) and the ground truth FFR value. In some embodiments, the loss function L may be a difference (e.g., L-1, L-2, etc.) between the generated prediction result y(t) and the ground truth FFR value.

In some embodiments, for the prediction of FFR, the input x(t) may be the images, image patches, masks, or features for points along the coronary artery tree. In some embodiments, various learning models such as CNN, FCN, MLP, or other method may be applied by the unconstrained derivative unit 403a to encode the input information. In some embodiments, the intermediate variable may be defined as a derivative function of FFR, or simply the drop of FFR relative to the previous upstream location along the vessel tree.

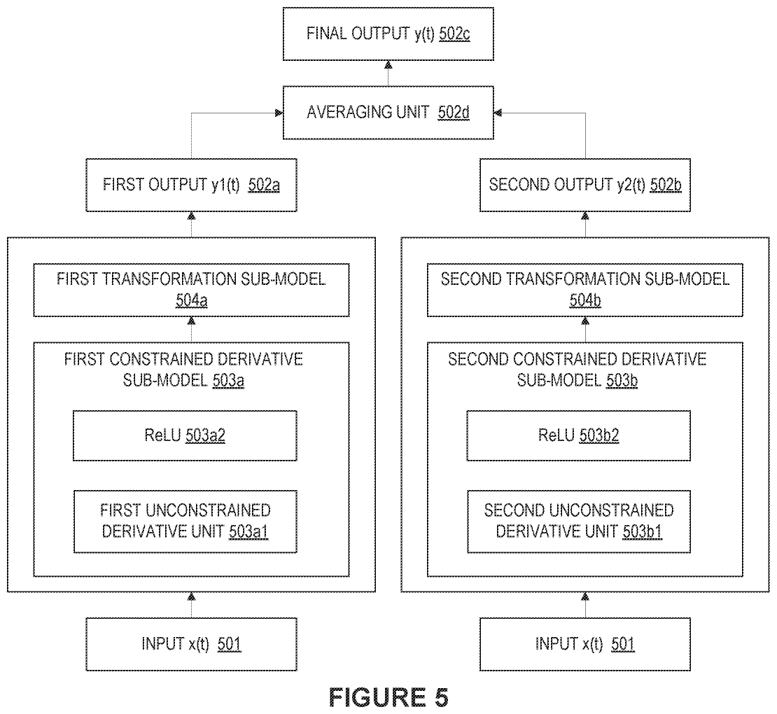

FIG. 5 illustrates a schematic diagram of another example of FFR prediction model according to an embodiment of the present disclosure. In some embodiments, the physical parameter to be predicted by the FFR prediction model is a single physical parameter, i.e., a single FFR for an individual position along the vessel tree, and the prior information of the bound range of the physical parameter is taken into account. Particularly, the bound range of a single FFR has a lower limit as 0 and an upper limit as 1.

As shown in FIG. 5, the FFR prediction model may include two parallel modeling branches, with the left one defined for the lower limit of the bound range while the right one defined for the upper limit of the bound range. For the left branch, a first intermediate variable may be defined based on subtracting the lower limit of the bound range from the FFR; and for the right branch, a second intermediate variable may be defined by subtracting the FFR from the upper limit of the bound range.

In some embodiments, the input x(t) 501, which may be image patch(es), feature vector(s), etc., may be fed into a first constrained subtraction sub-model 503a and a second constrained subtraction sub-model 503b. In some embodiments, the first constrained subtraction sub-model 503a may include a first unconstrained subtraction unit 503a1 and a ReLU 503a2 as the corresponding constraint function (also working as the activation function at the end of the learning model). The first unconstrained subtraction unit 503a1 may be built based on any one of CNN, MLP, etc., and may be configured to model and determine the difference between the FFR value and the lower limit (e.g., 0). The difference may then be mapped by the ReLU 503a2 into a non-negative range, to enforce the prior information associated with the lower limit. The ReLU 503a2 may output and feed the non-negative difference between the FFR value and the lower limit into a first transformation sub-model 504a. The first transformation sub-model 504a may be built based on a subtraction, e.g., an inverse operation to that performed by the first unconstrained subtraction unit 503a1, to obtain the FFR value therefrom as a first output y1(t) 502a.

Similarly, in the right branch for the upper limit, the second constrained subtraction sub-model 503b may include a second unconstrained subtraction unit 503b1 and a ReLU 503b2 as the corresponding constraint function (also working as the activation function at the end of the learning model). The second unconstrained subtraction unit 503b1 may be built based on any one of CNN, MLP, etc., and may be configured to model and determine the difference between the upper limit (e.g., 1) and the FFR value. The difference may then be mapped by the ReLU 503b2 into a non-negative range, to enforce the prior information associated with the upper limit. The ReLU 503b2 may output and feed the non-negative difference between the upper limit and FFR value into a second transformation sub-model 504b. Like the first transformation sub-model 504a, the second transformation sub-model 504b may also be built based on a subtraction, e.g., an inverse operation to that performed by the second unconstrained subtraction unit 503b1, to obtain the FFR value therefrom as a second output y2(t) 502b.

Both the first output y1(t) 502a and the second output y2(t) 502b may be utilized to obtain the final output y(t) 502c as the finally predicted FFR value. As an example, averaging operation may be performed by an averaging unit 502d with respect to the first output y1(t) 502a and the second output y2(t) 502b, to obtain the final output y(t) 502c. In some embodiments, other operations, such as minimization operation, etc., may be adopted to take both the first output y1(t) 502a and the second output y2(t) 502b into account to obtain the finally predicted FFR value.

Although FIG. 5 illustrates a parallel framework including one branch on the lower limit and the other branch on the upper limit, it is only an example. In some embodiments, either of the two branches may work independently. Besides, although FIG. 4 and FIG. 5 illustrate the transformation sub-models to be external to their respective constrained derivative sub-models, it is contemplated that these sub-models can be grouped into one model. Also, in some embodiments, the prior information may include both the non-decreasing monotonicity and the bound range, both of which can be applied as constraints. For example, the constrained derivative sub-model 403 shown in FIG. 4, and the first and second constrained subtraction sub-models 503a and 503b shown in FIG. 5, may be grouped within one FFR prediction model.

In some embodiments, the prior information of convex shape of the profile of the sequence of physical parameters may be adopted and enforced in the learning model. Accordingly, the intermediate variable may be defined based on the second order derivative, the activation function (such as but not limited to RELU) may be adopted at the end of the learning model, and the transformation function may be based on indefinite integration, to recover the physical parameters to be predicted from the output of the intermediate sub-model, i.e., the predicted second order derivatives of the sequence of physical parameters.

In the above embodiments, the coronary artery is used as an example of vessel, however, it is contemplated that the vessel may be any one of coronary artery, carotid artery, abdominal aorta, cerebral vessel, ocular vessel, and femoral artery, etc.

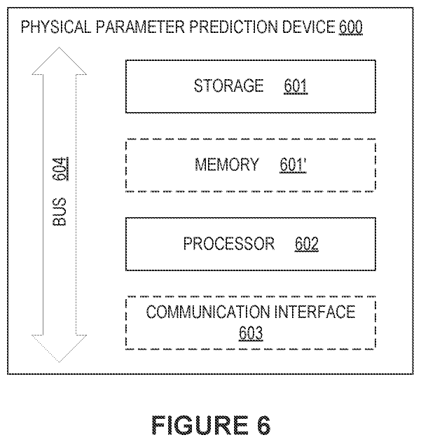

FIG. 6 illustrates a schematic block diagram of a physical parameter prediction device 600, which is used for predicting physical parameter based on the input physical information according to an embodiment of the present disclosure. As shown in FIG. 6, the physical parameter prediction device 600 may include a communication interface 603, a processor 602, a memory 601′, a storage 601, and a bus 604, and may also include a display. The communication interface 603, the processor 602, the memory 601′, and the storage 601 may be connected to the bus 604 and may communicate with each other through the bus 604.

The storage 601 may be configured to load or store the intermediate sub-model(s) according to any one or more embodiments of present disclosure, including, e.g., the constrained intermediate sub-models and transformation sub-models. The processor 602 may be configured to predict an intermediate variable based on the input physical information with the intermediate sub-model; and transform the intermediate variable predicted by the intermediate sub-model to the physical parameter with the transformation sub-model.

In some embodiments, the processor 602 may be a processing device including one or more general processing devices, such as a microprocessor, a central processing unit (CPU), a graphics processing unit (GPU), and so on. More specifically, the processor may be a complex instruction set computing (CISC) microprocessor, a reduced instruction set computing (RISC) microprocessor, a very long instruction word (VLIW) microprocessor, a processor running other instruction sets, or a processor that runs a combination of instruction sets. The processor may also be one or more dedicated processing devices, such as an application specific integrated circuit (ASIC), a field programmable gate array (FPGA), a digital signal processor (DSP), a system on a chip (SoC), etc.

The storage 601 may be a non-transitory computer-readable medium, such as read only memory (ROM), random access memory (RAM), phase change random access memory (PRAM), static random access memory access memory (SRAM), dynamic random access memory (DRAM), electrically erasable programmable read-only memory (EEPROM), other types of random-access memory (RAM), flash disks or other forms of flash memory, cache, register, static memory, compact disc read only memory (CD-ROM), digital versatile disk (DVD) or other optical memory, cassette tape or other magnetic storage devices, or any other possible non-transitory medium used to store information or instructions that can be accessed by computer equipment, etc. The instructions stored on the storage 601, when executed by the processor 602, may perform the method for predicting a physical parameter based on the input physical information according to any embodiment of present disclosure. In some embodiments, the physical parameter prediction device 600 may also perform the model training function, and accordingly, the storage 601 may be configured to load training dataset of the physical information annotated with the physical parameter, and the processor 602 may be configured to collectively train the intermediate sub-model and the transformation sub-model based on loaded training dataset.

In some embodiments, physical parameter prediction device 600 may further include a memory 601′, which may be configured to load the intermediate sub-model(s) according to any one or more embodiments of present disclosure. The processor 602 may be communicatively coupled to the memory 601′ and configured to execute computer executable instructions stored thereon, to perform a method for predicting a physical parameter based on the input physical information according to any embodiment of present disclosure.

In some embodiments, the memory 601′ may be a non-transitory computer-readable medium, such as read only memory (ROM), random access memory (RAM), phase change random access memory (PRAM), static random access memory access memory (SRAM), dynamic random access memory (DRAM), electrically erasable programmable read-only memory (EEPROM), other types of random access memory (RAM), flash disks or other forms of flash memory, cache, register, static memory, or any other possible medium used to store information or instructions that can be accessed and executed by computer equipment, etc.

In some embodiments, physical parameter prediction device 600 may further include a communication interface 603. In some embodiments, the communication interface 603 may include any one of a network adapter, a cable connector, a serial connector, a USB connector, a parallel connector, a high-speed data transmission adapter (such as optical fiber, USB 3.0, Thunderbolt interface, etc.), a wireless network adapter (Such as WiFi adapter), telecommunication (3G, 4G/LTE, 5G, etc.) adapters, etc.

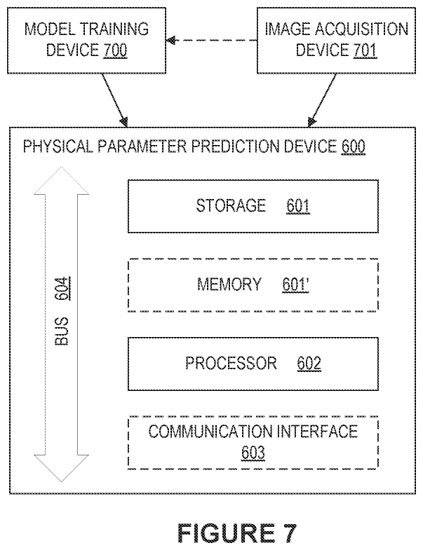

FIG. 7 illustrates a schematic block diagram of a system for predicting physical parameter based on the input physical information according to an embodiment of the present disclosure. As shown, the system may comprise a physical parameter prediction device 600, a model training device 700, and an image acquisition device 701. The details of the physical parameter prediction device 600 has already mentioned as above, and thus are not repeated here.

Specifically, the image acquisition device 701 may include any one of normal CT, normal MRI, functional magnetic resonance imaging (such as fMRI, DCE-MRI, and diffusion MRI), cone beam computed tomography (CBCT), positron emission tomography (PET), Single-photon emission computed tomography (SPECT), X-ray imaging, optical tomography, fluorescence imaging, ultrasound imaging and radiotherapy field imaging, etc.

In some embodiments, the model training device 700 may be configured to train the physical parameter prediction model (for example, the unconstrained intermediate sub-model therein), and transmit the trained physical parameter prediction model to the physical parameter prediction device 600 for predicting physical parameter based on the input physical information according to any embodiment of present disclosure, by using the trained physical parameter prediction model. In some embodiments, the model training device 700 and the physical parameter prediction device 600 may be implemented by a single computer or processor.

In some embodiments, the physical parameter prediction device 600 may be a special purpose computer or a general-purpose computer. For example, the physical parameter prediction device 600 may be a computer customized for a hospital to perform image acquisition and image processing tasks, or may be a server in the cloud.

The physical parameter prediction device 600 may be connected to the model training device 700, the image acquisition device 701, and other components through the communication interface 603. In some embodiments, the communication interface 603 may be configured to receive a trained physical parameter prediction model from the model training device 700, and may also be configured to receive medical images from the image acquisition device 701, such as a set of images of vessels.

In some embodiments, the storage 601 may store a trained model, prediction result of the physical parameter, or the intermediate information generated during the training phase or the prediction phase, such as feature information generated while executing a computer program. In some embodiments, the memory 601′ may store computer-executable instructions, such as one or more image processing (such as physical parameter prediction) programs. In some embodiments, each unit, function, sub-model, and model may be implemented as applications stored in the storage 601, and these applications can be loaded to the memory 601′, and then executed by the processor 602 to implement corresponding processes.

In some embodiments, the model training device 700 may be implemented using hardware specially programmed by software that executes the training process. For example, the model training device 700 may include a processor and a non-transitory computer readable medium similar to the physical parameter prediction device 600. The processor implements training by executing executable instructions for the training process stored in a computer-readable medium. The model training device 700 may also include input and output interfaces to communicate with the training database, network, and/or user interface. The user interface may be used to select training data sets, adjust one or more parameters in the training process, select or modify the framework of the learning model, etc.

Another aspect of the disclosure is directed to a non-transitory computer-readable medium storing instructions which, when executed, cause one or more processors to perform the methods, as discussed above. The computer-readable medium may include volatile or non-volatile, magnetic, semiconductor-based, tape-based, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

Various modifications and changes can be made to the disclosed method, device, and system. In view of the description and practice of the disclosed system and related methods, other embodiments can be derived by those skilled in the art. Each claim of the present disclosure can be understood as an independent embodiment, and any combination between them can also be used as an embodiment of the present disclosure, and it is considered that these embodiments are all comprised in the present disclosure.

It is intended that the description and examples are to be regarded as exemplary only, with the true scope being indicated by the appended claims and their equivalents.

What is claimed is:

1. A method for predicting a physical parameter based on input physical information, comprising:
predicting, by a processor, an intermediate variable based on the input physical information with an intermediate sub-model, which incorporates a constraint on the intermediate variable according to prior information of the physical parameter; and
transforming, by the processor, the intermediate variable predicted by the intermediate sub-model to the physical parameter with a transformation sub-model.

2. The method of claim 1, wherein the intermediate sub-model is based on a learning model, the transformation sub-model is a preset function, and the intermediate sub-model and the transformation sub-model collectively trained with training dataset comprising sample physical information annotated with corresponding ground truth physical parameter.

3. The method of claim 1, wherein the intermediate sub-model is configured to predict an unconstrained intermediate variable and apply the constraint to the unconstrained intermediate variable to predicted the intermediate variable.

4. The method of claim 1, wherein the prior information of the physical parameter comprises a profile tendency of a profile of the physical parameter or a bound range of the physical parameter in a temporal domain or a spatial domain.

5. The method of claim 4, wherein the profile tendency comprises any one of a monotonicity of profile change, a periodicity of profile change, a convex shape of the profile, and a concave shape of the profile.

6. The method of claim 1, wherein the intermediate sub-model is based on a learning model, and the constraint comprises an activation function.

7. The method of claim 6, wherein the physical parameter to be predicted includes a sequence of physical parameters, the prior information of the physical parameter is a monotonicity of profile change of the sequence of physical parameters, the intermediate variable is a derivative of the sequence of physical parameters, the constraint comprises an activation function, and the transformation function is an integral function.

8. The method of claim 7, wherein the sequence of physical parameters comprise vessel parameters at a sequence of positions in a vessel.

9. The method of claim 8, wherein the vessel has a structure of a vessel tree, or a vessel path.

10. The method of claim 6, wherein the physical parameter to be predicted is a single physical parameter, the prior information of the physical parameter is a bound range of the physical parameter, the intermediate variable is determined by subtracting a lower limit of the bound range from the physical parameter or subtracting the physical parameter from a upper limit of the bound range, the constraint is an activation function, and the transformation function is a subtraction.

11. The method of claim 6, wherein the physical parameter to be predicted comprises a sequence of physical parameters, the prior information of the physical parameter is a convex shape of a profile of the sequence of physical parameters, the intermediate variable is a second order derivative of the sequence of physical parameters, the constraint is an activation function, and the transformation function is an indefinite integration.

12. A device for predicting a physical parameter based on input physical information, comprising:
a storage configured to load or store an intermediate sub-model and a transformation sub-model; and
a processor configured to:
predict an intermediate variable based on the input physical information with the intermediate sub-model, which incorporates a constraint on the intermediate variable according to prior information of the physical parameter; and
transform the intermediate variable predicted by the intermediate sub-model to the physical parameter with the transformation sub-model.

13. The device of claim 12, wherein the intermediate sub-model is based on a learning model, the transformation sub-model is a preset function, and the intermediate sub-model and the transformation sub-model collectively trained with training dataset comprising sample physical information annotated with corresponding ground truth physical parameter.

14. The device of claim 12, wherein the intermediate sub-model is configured to predict an unconstrained intermediate variable and apply the constraint to the unconstrained intermediate variable to predicted the intermediate variable.

15. The device of claim 12, wherein the prior information of the physical parameter comprises a profile tendency of a profile of the physical parameter or a bound range of the physical parameter in a temporal domain or a spatial domain.

16. The device of claim 15, wherein the profile tendency comprises any one of a monotonicity of profile change, a periodicity of profile change, a convex shape of the profile, and a concave shape of the profile.

17. The device of claim 12, wherein the intermediate sub-model is based on a learning model, and the constraint comprises an activation function.

18. The device of claim 17, wherein the physical parameter to be predicted includes a sequence of physical parameters, the prior information of the physical parameter is a monotonicity of profile change of the sequence of physical parameters, the intermediate variable is a derivative of the sequence of physical parameters, the constraint comprises an activation function, and the transformation function is an integral function.

19. The device of claim 18, wherein the sequence of physical parameters comprise vessel parameters at a sequence of positions in a vessel.

20. A non-transitory computer-readable medium having computer-executable instructions stored thereon, wherein the computer-executable instructions, when executed by a processor, perform a method for predicting a physical parameter based on input physical information, the method comprising:
predicting an intermediate variable based on the input physical information with an intermediate sub-model, which incorporates a constraint on the intermediate variable according to prior information of the physical parameter; and
transforming the intermediate variable predicted by the intermediate sub-model to the physical parameter with a transformation sub-model.
